(19) United States
(12) Patent Application Publication
(10) Pub. No.: US 2019/0184666 A1
(43) Pub. Date: Jun. 20, 2019

(54) APPARATUS FOR RECOVERING SOLVENT FROM BIOMASS

(71) Applicants: Jacqueline McGrane, Black Hawk, CO (US); Randall Davis, Thornton, CO (US); Saman Razani, Palm Desert, CA (US)

(72) Inventors: Jacqueline McGrane, Black Hawk, CO (US); Randall Davis, Thornton, CO (US); Saman Razani, Palm Desert, CA (US)

(21) Appl. No.: 15/846,092

(22) Filed: Dec. 18, 2017

(51) Int. Cl. B30B 9/06; A61K 36/00; B01D 11/02; F26B 5/14

(52) U.S. Cl. CPC B30B 9/06; A61K 36/00; A61K 2236/50; F26B 5/14; A61K 2236/33; B01D 11/0219

(57) ABSTRACT

Apparatuses for recovering solvent from biomass are disclosed herein. In one example, the apparatus can include a mechanical press configured to exert pressure on wetted plant material to force liquid solvent from the plant matter so the solvent can be collected for reuse. The apparatus can also be configured to flow pressurized gas through the wetted plant matter, thereby forcing liquid solvent from the plant material so the solvent can be collected for reuse.

### FIELD

This disclosure relates to apparatuses for recovering solvent from biomass. More specifically, this disclosure relates to apparatuses for recovering solvent from biomass during an essential oil extraction process.

### BACKGROUND

Solvents, such as ethanol, can be used to extract essential oils from plant matter. Examples of plant matter that contain useful essential oils include lavender flowers, eucalyptus leaves, peppermint leaves, tea tree leaves, jojoba seeds, rose petals, cannabis flowers, and jasmine flowers. Essential oils are used in a wide variety of applications, including as additives in household cleansers and personal care products (e.g. shampoos, lotions, facial cleansers) and in pain relief treatments.

In an essential oil extraction process using a quick-wash ethanol technique, plant matter (i.e. biomass) is submerged in ethanol for a period of time. While submerged, the solvent removes essential oils from the plant matter. After the essential oils have been removed from the plant matter, the spent plant matter is removed from the solvent and discarded. The solution of solvent and essential oils is then processed to isolate the essential oils.

The spent plant matter that is removed from the solvent is typically wetted with solvent. In a small operation, such as when extracting essential oils from flowers at home, the amount of solvent remaining in the spent plant matter may be relatively small (e.g. 20 percent of the total amount of solvent used) and, consequently, of relatively little value. Therefore, it may not make economic sense to spend time and effort attempting to recover the remaining solvent from the plant matter before discarding it. However, when operating a large scale essential oil extraction process, where many large batches of plant matter are processed every hour and large volumes of solvent are used (e.g. to extract essential oils for large batches of personal care products, such as perfumes or shampoos), it can be desirable to spend time and effort recovering solvent from the spent plant matter for reuse. By recovering as much solvent as possible, the process operator reduces the amount of solvent that has to be purchased to sustain the process and also significantly reduces the amount of solvent that must be disposed of (e.g. trucked away) with the spent plant matter.

An apparatus is needed to increase the percentage of solvent that can be quickly, easily, and affordably recovered from spent plant matter before it is discarded during an essential oil extraction process.

### BRIEF DESCRIPTION OF THE DRAWINGS

FIG. 1 shows a front right perspective view of an apparatus for recovering solvent from biomass.

FIG. 2 shows a front left perspective view of the apparatus of FIG. 1.

FIG. 3 shows a perspective view of a portion of the apparatus of FIG. 1, including an upper press member, lower press member, and biomass receptacle supported by the lower press member.

FIG. 4 shows a perspective view of a portion of the apparatus of FIG. 1, including an upper press member, lower press member, and biomass receptacle supported by the lower press member.

FIG. 5 shows a bottom perspective view of a biomass receptacle with a plurality of thru holes.

FIG. 6 shows a bottom perspective view of a portion of the apparatus of FIG. 1, including an upper press member and lower press member with the biomass receptacle removed.

FIG. 7 shows a top perspective view of a portion of the apparatus of FIG. 1, including an upper press member, gas injection system, and lower press member with the biomass receptacle removed to expose a drainage opening.

FIG. 8 shows a top perspective view of a portion of the apparatus of FIG. 1, including an upper press member, gas injection system, and lower press member with the biomass receptacle removed to expose a drainage opening.

FIG. 9 shows a side cross-sectional view of an apparatus for recovering solvent from biomass, the apparatus in an open position with the upper press member spaced apart from the biomass receptacle.

FIG. 10 shows a side cross-sectional view of the apparatus of FIG. 9, the apparatus in a closed position with the upper press member sealed against the biomass receptacle.

FIG. 11 shows a step of soaking biomass in solvent to extract essential oils from the biomass.

FIG. 12 shows a step of transferring the biomass from the vessel containing solvent to a solvent recovery apparatus using a transfer plate.

FIG. 13 shows a step of placing the biomass, which is wetted with solvent, in a biomass receptacle of a solvent recovery apparatus.

FIG. 14 shows the steps of applying compressive force to the biomass by transitioning the apparatus from an open position to a closed position and applying gas pressure via a gas injection system.

FIG. 15 shows a compressed gas delivery system fluidly connected to a gas injection system of a solvent recovery apparatus.

FIG. 16 shows a portion of an essential oil extraction system including a dunk tank, apparatus for recovering solvent from biomass, and a conical storage vessel.

### SUMMARY

In one example, an apparatus for recovering liquid solvent from biomass can include a biomass receptacle configured to receive a mixture of biomass and liquid solvent, a means for exerting a compressive force on the mixture of biomass and liquid solvent while the mixture is positioned in the biomass receptacle, a means for flowing pressurized gas through the mixture while the mixture is positioned in the biomass receptacle, and a means for collecting the liquid solvent that exits the biomass receptacle.

In another example, an apparatus for recovering liquid solvent from wetted biomass can include a biomass receptacle having an inner surface, an outer surface, and a plurality of holes extending through the biomass receptacle from the inner surface to the outer surface where the inner surface of the biomass receptacle defines an inner volume. The apparatus can include a lower press member having a support surface configured to receive and support the biomass receptacle, an upper press member having a lower surface configured to seal against a rim surface of the biomass receptacle, and an actuator configured to transition the apparatus from an open position to a closed position, where the inner volume of the biomass receptacle is accessible when the apparatus is in the open position, and where the lower surface of the upper press member seals against the rim surface of the biomass receptacle when the apparatus is in the closed position. The apparatus can include a gas injection system configured to deliver pressurized gas to gas inlets in the lower surface of the upper press member. The gas injection system can be configured to deliver gas at a pressure of 15-80 psi to the gas inlets in the lower surface of the upper press member. The actuator can be configured to apply a compressive force of 10-50, 25-75, 50-100, 75-150, or 100-200 psi between the upper press member and the biomass receptacle when the apparatus is in the closed position. A portion of the upper press member can occupy a portion of the inner volume of the biomass receptacle when the apparatus is in the closed position, thereby decreasing available space within the inner volume for a mixture of biomass and solvent and allowing the upper press to compress the biomass and squeeze solvent from the biomass. The lower surface of the upper press member can be a convex surface that occupies a portion of the inner volume of the biomass receptacle when the apparatus is in the closed position, thereby decreasing available space within the inner volume for a mixture of biomass and solvent and allowing the upper press to compress the biomass and squeeze solvent from the biomass. The lower press member can include a drainage surface and a drainage opening fluidly connected to the drainage surface. The apparatus can include a gap between the outer surface of the biomass receptacle and the drainage surface of the lower press member where the gap is configured to permit drainage of liquid solvent from the holes in the biomass receptacle to the drainage opening in the lower press member when the apparatus is in the closed position. At least one of the plurality of holes in the biomass receptacle can have a diameter of 0.125-0.375 in. The gas injection system can include a gas manifold fluidly connected to one or more gas passageways, where the one or more gas passageways are fluidly connected to the gas inlets in the upper press member. Each gas passageway can be configured to deliver pressurized gas into the inner volume of the biomass receptacle when the apparatus is in the closed positioned and pressurized gas is supplied to the manifold. The apparatus can include a seal between the lower surface of the upper press member and the rim surface of the biomass receptacle. The actuator can be a pneumatic actuator or a hydraulic actuator.

In another example, an apparatus for recovering liquid solvent from biomass can include a press having an upper press member, a lower press member, and an actuator, where the actuator is configured to reduce the distance between the lower press member and the upper press member. The apparatus can include a biomass receptacle positioned between the lower press member and the upper press member, where the lower press member and upper press member together are configured to exert a compressive force on the biomass receptacle when the apparatus is in a closed position. The apparatus can include a gas injection system configured to deliver pressurized gas to an inner volume of the biomass receptacle when the apparatus is in the closed position. The apparatus can include a drainage opening configured to allow liquid solvent to flow from the biomass receptacle when the apparatus is in the closed position and a compressive force is exerted on a mixture of biomass and solvent present in the biomass receptacle. The actuator can be configured to reduce the distance between the lower press member and the upper press member by advancing the lower press member toward the upper press member. The actuator can be configured to reduce the distance between the lower press member and the upper press member by advancing the upper press member toward the lower press member. The biomass receptacle can have an inner surface, an outer surface, and a plurality of openings extending from the inner surface to the outer surface. The inner surface of the biomass receptacle can be hemispherical. The upper press member can have a convex hemispherical surface that is configured to exert a compressive force on a mixture of biomass and solvent when the mixture is located in the concave hemispherical biomass receptacle. The apparatus can include a flexible receptacle having an interior bag and an exterior bag, where the interior bag is configured to insert within the exterior bag, and where the exterior bag comprises a durable fabric.

### DETAILED DESCRIPTION

Apparatuses and methods for extracting solvent from biomass are disclosed herein. In a preferred embodiment, the apparatus 100 can include a physical press configured to exert pressure on wetted plant mater to force liquid solvent from the plant matter so the solvent can be collected for reuse. The apparatus can also be configured to apply pressurized gas to the wetted plant matter, thereby passing pressurized gas through the wetted plant matter and carrying liquid solvent away from the plant matter so the solvent can be collected for reuse. Because the apparatus does not rely on heat or vacuum to recover solvent, it can be less expensive to manufacture and operate than existing solvent recovery apparatuses.

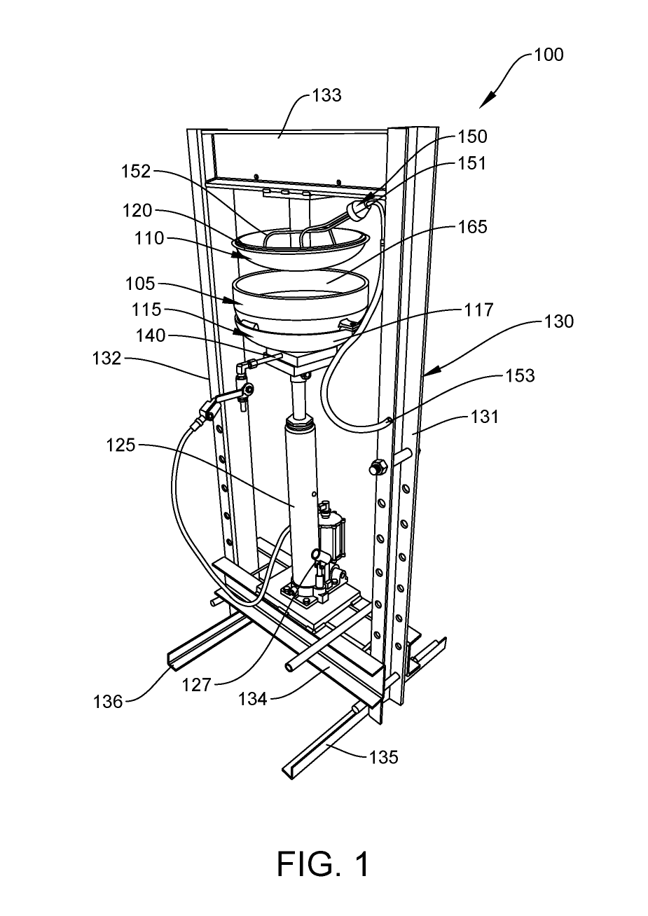

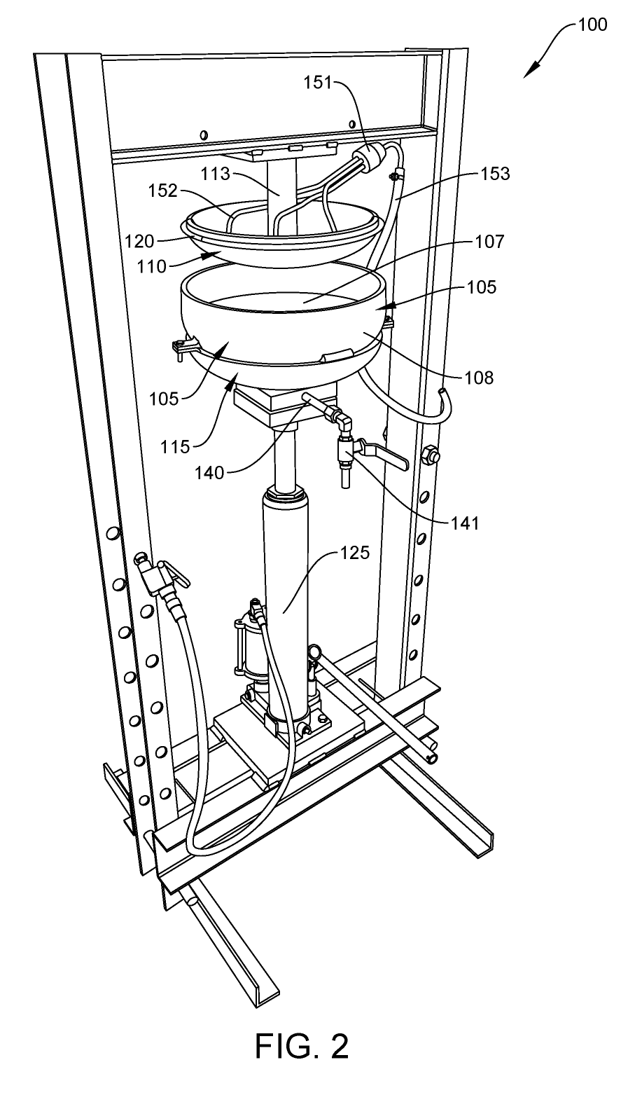

An apparatus 100 for recovering solvent from biomass can include a supporting frame 130. In one example, shown in FIGS. 1 and 2, the frame 130 can include a first upright member 131, a second upright member 132, and an upper cross member 133 connecting an upper portion of the first upright member 131 to an upper portion of the second upright member 132. The frame 130 can include a lower cross member 133 connecting a lower portion of the first upright member 131 to a lower portion of the second upright member 132. The frame 130 can include a first stabilizing member 135 and a second stabilizing member 136 that extend outwardly to stabilize the apparatus 100 and prevent the apparatus from tipping over during use.

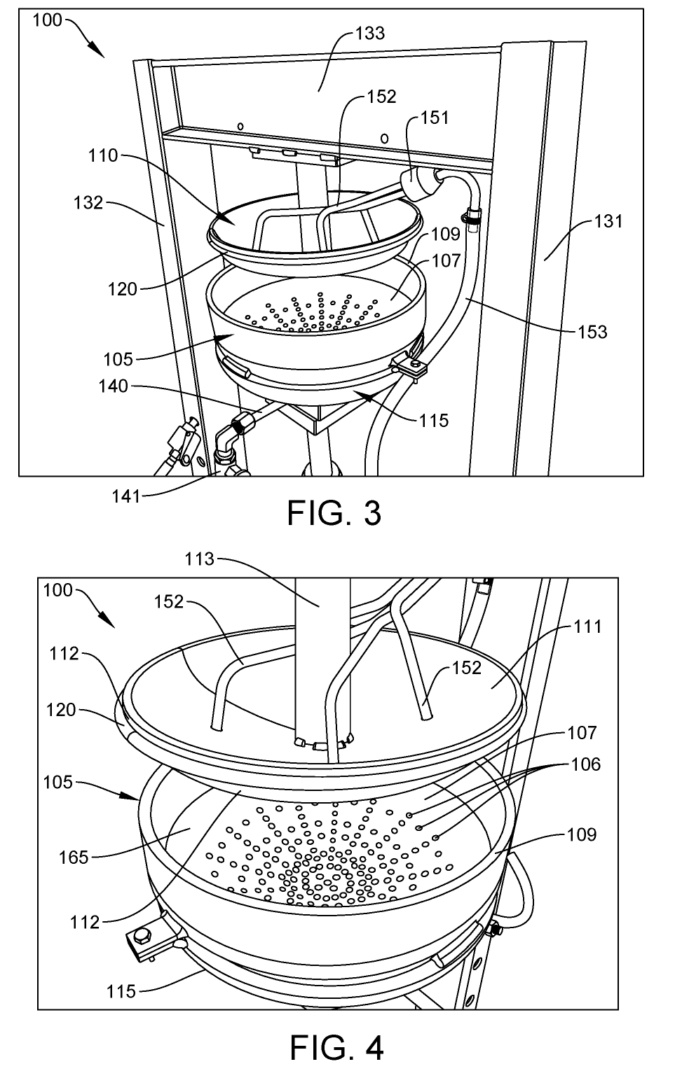

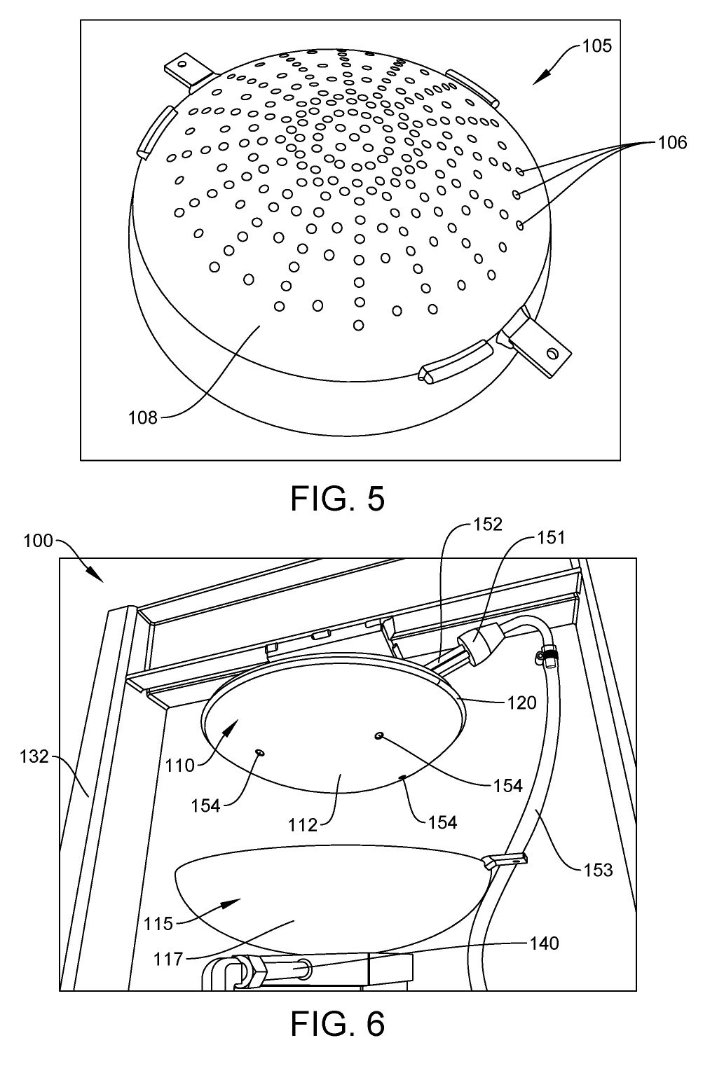

The apparatus can include a biomass receptacle 105. An example of the biomass receptacle 105 is shown in FIGS. 1-5. The biomass receptacle 105 can have an inner surface 107 and an outer surface 108. The biomass receptacle 105 can have a plurality of openings 106 passing from the inner surface to the outer surface, as shown in FIG. 5, to form a perforated basket. The openings 106 can be holes with diameters that are large enough to permit the liquid solvent to easily flow from the wetted biomass to the drainage surface of the lower press 115 during operation of the apparatus. However, it is also desirable for the diameters of the holes to be small enough to prevent the outer material of the flexible receptacle 160 from being drawn into the holes and potentially damaged (e.g. torn or irreversibly deformed). In some examples, the holes in the biomass receptacle 105 can have a diameter of about 0.0625-0.25, 0.125-0.375, 0.25-0.5, 0.375-0.525, 0.125, 0.25-0.5 in. In a preferred embodiment, the holes can have a diameter of about 0.25 in. The edges of the holes 106 can be sanded or polished smooth to avoid tearing the outer fabric of the flexible receptacle 160 during operation of the physical press 100 and gas injection system 150 of the apparatus. In one example, the holes 106 can be formed with a water jet and the inner surface 107 of the biomass receptacle 105 can be wet sanded or polished. In one example, as shown in FIG. 5, the holes 106 can be spaced apart and arranged in a uniform radial pattern to allow for uniform drainage of solvent through the biomass receptacle 105.

The biomass receptacle 105 can be made of a rigid material capable of withstanding compressive force applied by components of the apparatus 100 without deflecting. In some examples, the biomass receptacle 105 can be made of a food-safe material (e.g. stainless steel) and have a thickness of at least 0.125, 0.25, 0.375, or 0.5 in and preferable about 0.375 in.

The biomass receptacle 105 can have a diameter of at least 6, 12, 18, 24, or 30 in. In one example, the inner surface 107 of the biomass receptacle 105 can be curved (e.g. concave). In one example, the inner surface 107 of the biomass receptacle 105 can be hemispherical, similar to a well cap used to seal pressure vessels. Curvature of the inner surface 107 may be desirable for several reasons. First, the curved inner surface 107 may provide more uniform pressure distribution during a physical pressing step, thereby improving performance and increasing longevity of the biomass receptacle. The curved inner surface 107 may serve to self-align the biomass receptacle 105 with the upper press member (plunger) 110 during operation, thereby improving performance and reducing downtime for mechanical adjustments. The curved surface may improve solvent recovery yields by containing the solvent, utilizing gravity to recover solvent, and not allowing solvent to escape at a perimeter of the biomass receptacle, which can occur in a flat press.

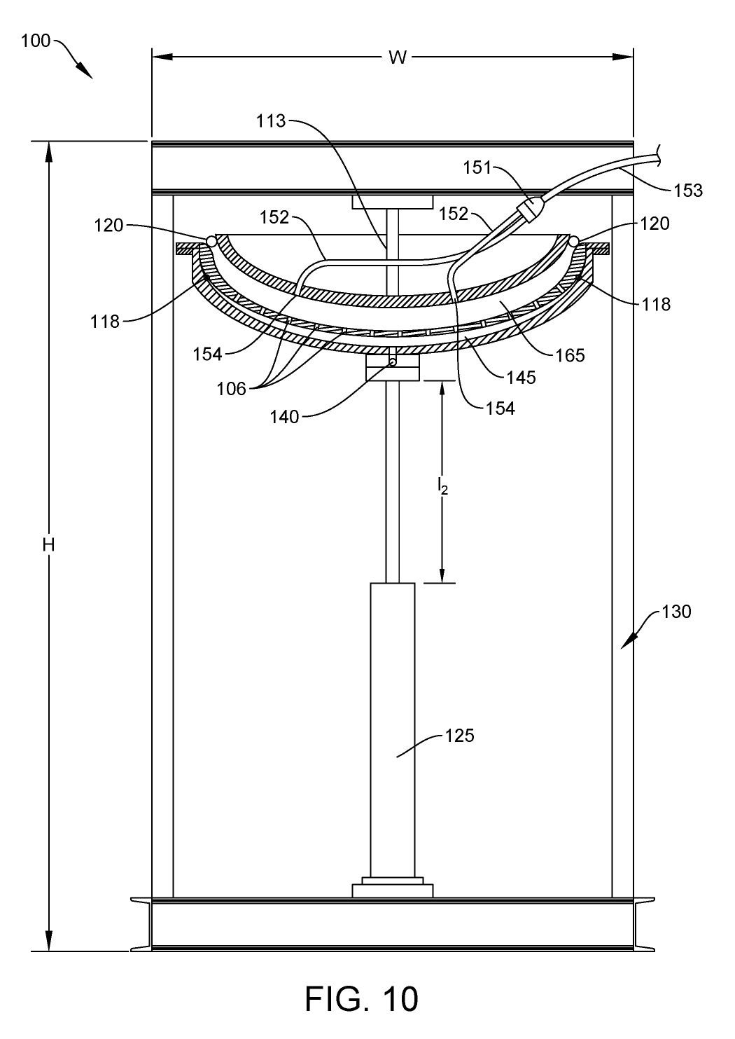

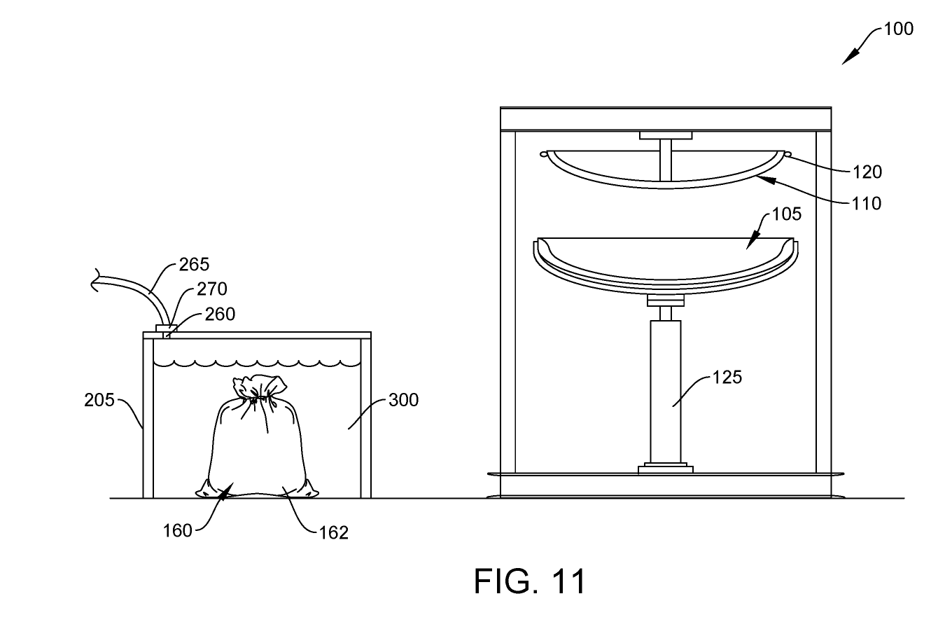

The apparatus 100 can include an upper press member 110 and a lower press member 115 and a means for advancing the upper and lower press members toward each other to exert compressive force on biomass 400 positioned within the biomass receptacle 105, thereby squeezing the biomass and causing solvent 300 to exit the biomass and allowing the solvent to be recovered for reuse. In the example shown in FIG. 1, the upper press member 110 can remain stationary, and the lower press member 115 can move toward the upper press member to a closed position. In the closed position shown in FIG. 10, the lower press member 115 can exert a compressive force against the upper press member 110 via the biomass receptacle 105. In another example, the lower press member 115 can remain stationary, and the upper press member 110 can move toward the lower press member to a closed position. In yet another example, both the upper and lower press members can be movable toward each other.

In the example shown in FIG. 1, the upper press member 110 can be attached to the frame 130 of the apparatus by a rigid support member 113. The upper press member can include an upper surface 111, a lower surface opposite 112 the upper surface, and a rim surface 109 extending around a perimeter of the upper press member.

The upper press member (plunger) 110 can be made of a rigid material capable of withstanding compressive force without deflecting significantly. In some examples, the upper press member 110 can be made of a food-safe material (e.g. stainless steel) and have a thickness of at least 0.25, 0.375, or 0.5 in. The lower surface 112 of the upper press member 110 can be curved (e.g. convex). In one example, the lower surface 112 of the upper press member 110 can be hemispherical. Curvature of the lower surface 112 of the upper press member 110 may be desirable for several reasons. First, the curved lower surface 112 may provide more uniform pressure distribution thereby improving performance and increasing longevity of the upper press member 110. The curved lower surface 112 may serve to self-align the upper press member 110 with the biomass receptacle 105 during operation, thereby improving performance and reducing downtime for mechanical adjustments.

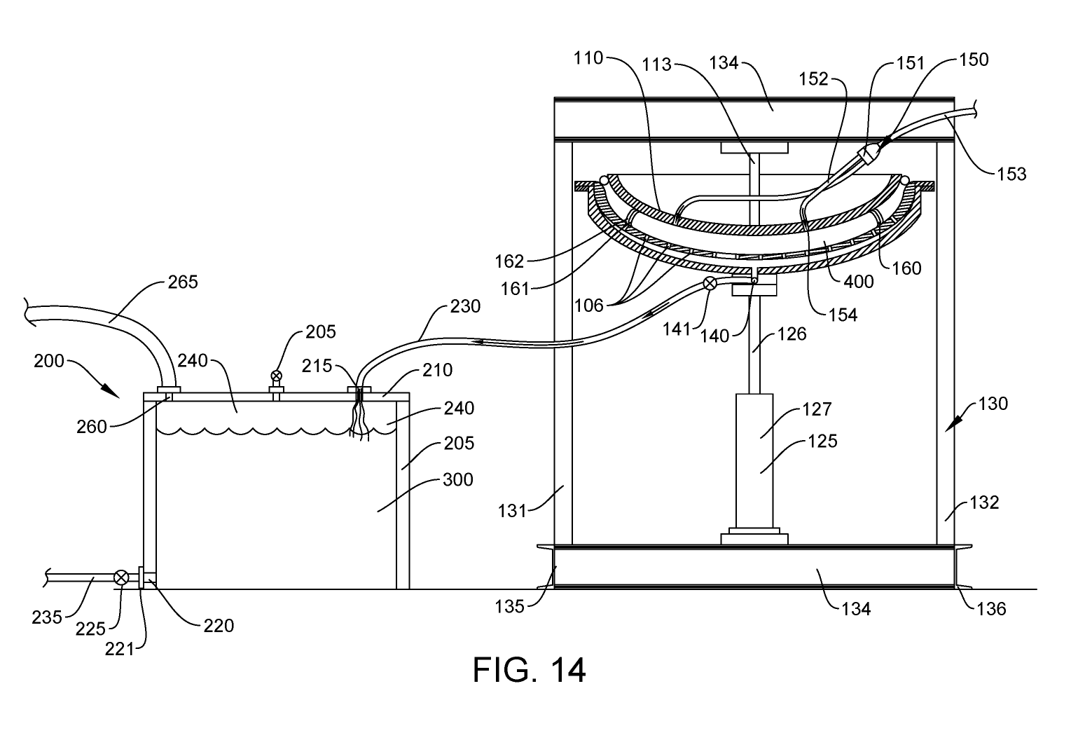

The solvent recovery apparatus 100 can include a gas injection system 105 capable of delivering compressed gas to biomass 400 within the apparatus. Flowing compressed gas through the biomass can significantly improve the percentage of solvent 300 that is recovered from the wetted biomass. In a preferred example, the biomass 400 can be subjected to physical pressing first and then, while the biomass remains subjected to physical pressing, compressed gas is applied to the biomass, as shown in FIG. 14. The compressed gas can flow through the biomass and entrain and transport liquid solvent through the openings in the biomass receptacle, where the solvent can then be collected and reused. In another example, the wetted biomass can first be exposed to a flow of compressed gas and then be physically pressed. In another example, the wetted biomass can be exposed to a flow of compressed gas while it is being physically pressed. In still another example, the biomass 400 can be subjected to physical pressing first and then, after the pressing is complete and the press is relaxed, compressed gas can be applied to the biomass.

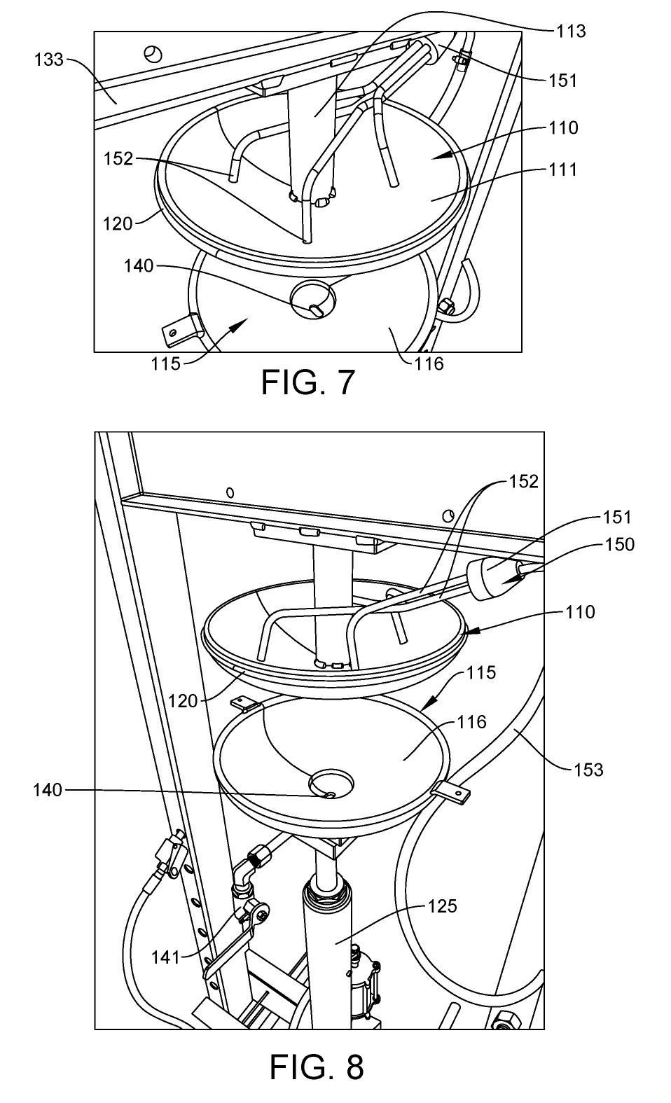

An example of a gas injection system 150 is shown in FIGS. 1-10. The gas injection system 150 can include a gas supply line 153. The gas supply line 153 can be fluidly connected to a gas manifold 151. The gas manifold 151 can be fluidly connected to a plurality of gas passageways 152 that deliver pressurized gas to corresponding gas inlets 154 in the lower surface 112 of the upper press member 110. FIG. 6 shows an upper press member 110 with four gas inlets 154 located equidistant from the center of the lower surface 112 of the upper press member, each gas inlet being located in about the center of a quadrant of the lower surface 112. In another example, the upper press member can have more than four, more than 6, or more than 8 gas inlets 154. Preferably, the upper press member 110 has at least one gas inlet 154 located near the center of its lower surface 112 and at least one gas inlet 154 located in each quadrant of its lower surface 112 to facilitate even distribution of compressed gas to wetted biomass 400 within the biomass receptacle 105.

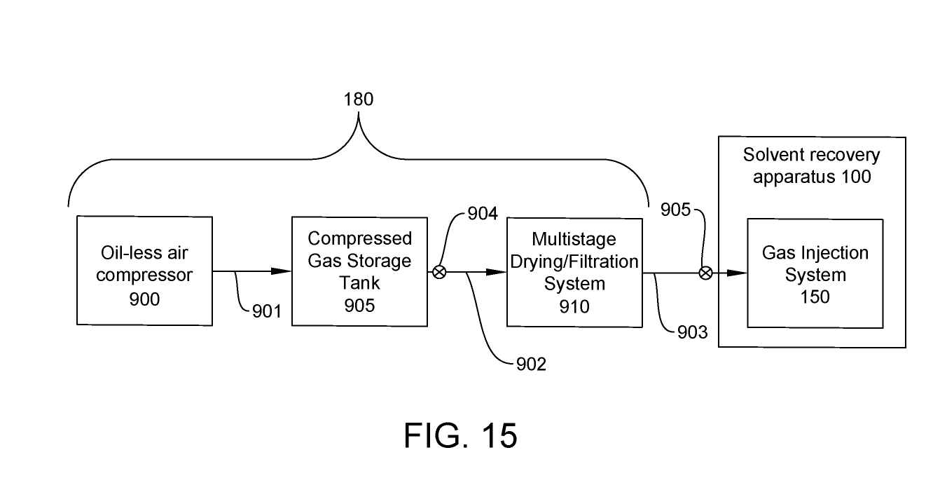

The gas injection system 150 can receive dry, clean compressed gas from a compressed gas supply system 180, as shown in FIG. 15. In some instances, the gas injection system 150 can deliver compressed gas, such as nitrogen, argon, or carbon dioxide, to the solvent recovery apparatus 100. In other instances, the gas injection system 150 can deliver clean, dry compressed air to the apparatus 100. To avoid contaminating the solvent 300 with water or particles, it is desirable to remove water vapor and unwanted particles from the compressed air prior to delivering the air to the gas injection system 150. The compressed gas delivery system 180 can be configured to remove water vapor and unwanted particles from the compressed gas before it reaches the gas injection system 150.

Since the essential oils that are extracted from the plant matter may be consumed by humans or used in personal care products, it is desirable to comply with food safety regulations and to use food-safe components in the solvent recovery process. Accordingly, the compressed gas that is used to purge solvent from the biomass during the gas injection process should be clean and free of unwanted particles.

A compressed gas supply system 180, as shown in FIG. 15, can supply clean, dry compressed gas to the gas injection system 150. The compressed gas supply system 180 can include an oil-less compressor 900 fluidly connected to a compressed gas storage tank 905 by a gas supply line 901. Using an oil-less compressor is desirable to avoid introducing lubricating oil mist into the compressed air during the compression stage. The storage tank 905 can serve as a reservoir of compressed gas. As compressed gas is drawn from the storage tank 905 for use by the gas injection system 150, the compressor 900 can cycle on to replenish the storage tank with compressed gas as needed to maintain a desired gas pressure within the tank. In one example, the compressor can be operated to maintain the storage tank at a pressure of about 40-80, 60-100, 80-120, or preferably about 60-80 psi. The compressed gas supply system 180 can include a gas supply line 902 extending between the gas storage tank 905 and a gas filtration system 910. In one example, the gas supply line 902 can be at least 50 feet in length to permit compressed air, which may be at an elevated temperature due to the compression process, to cool prior to reaching the gas filtration system 910. By increasing the length of the gas supply line 902, the residence time of the compressed gas traveling through the supply line is increased, which allows the gas sufficient time to cool, which can allow water vapor in the gas to condense so it can be more easily removed. A filter in the gas filtration system 910 can remove condensed water from the compressed gas. The gas filtration system can include at least one particulate filter to allow for unwanted particles to be removed from the compressed gas before it is delivered to the gas injection system 150 of the apparatus 100. Preferably, the gas filtration system can include two or more particulate filters connected in series to allow for progressively smaller unwanted particles to be removed from the compressed gas before it is delivered to the gas injection system 150 of the apparatus 100. In one example, the filtration system can be a four-stage air drying system, such as a model number U4060M-N04DG-MEP Four Stage Air Drying System from PneumaticPlus including a 10 micron particulate filter/regulator, 0.3 micron oil mist removing filter, a 0.01 micron coalescing filter, and a drain to permit draining of collected water. A gas supply line 903 can fluidly connect the gas filtration system 910 to the gas injection system 150 of the apparatus 100, as shown in FIG. 15.

The compressed gas delivery system 180 can include one or more flow control devices (e.g. 904, 905), such as valves or regulators, to control flow of pressurized gas through the compressed gas supply system 180. A first flow control device 904 can be located between the storage tank 905 and the gas filtration system 910. A second flow control device 910 can be located between the gas filtration system 910 and the gas injection system 150.

In one example, the compressed gas delivery system 180 can include at least one pressure regulator (e.g. 904, 905) located between the storage tank 905 and the gas injection system 150. The pressure regulator can allow the system to deliver compressed gas at any pressure at or below the pressure of gas in the storage tank 904. This can allow an operator of the gas injection system 150 to adjust the pressure based on certain factors, such as type of plant matter, level of homogenization of the plant material, and desired cycle time.

The upper press member 110 can include a seal 120 proximate the lower surface 112 of the upper press member, as shown in FIG. 4. The seal 120 can extend around a perimeter of the upper press member 110. To keep the seal 120 in place, it can be seated in an O-ring groove that extends around the perimeter of the upper press member 110. The seal 120 can allow the upper press member 110 to seal against a surface (e.g. a rim surface 109) of the biomass receptacle 105, as shown in FIG. 10, and form a gas-tight seal. The seal 120 can be a rubber (e.g. butyl rubber) O-ring, gasket, or any other suitable sealing member that is compatible with the solvent. The seal 120 can prevent pressurized gas that is delivered to the inner volume 165 of the biomass receptacle 105 by the gas injection system 150 from escaping from the inner volume at the junction between the upper press member 110 and the biomass receptacle. Instead, when the apparatus 100 is in the closed position, as shown in FIG. 14, the seal 120 ensures that compressed gas delivered to the inner volume 165 of the biomass receptacle 105 can only leave the inner volume of the biomass receptacle through the holes 106 in the biomass receptacle. The compressed gas flows downward through the holes 106 in the biomass receptacle 105 and drives the solvent to the drainage opening 140 where it can be recovered. In an alternate embodiment, the seal 120 can be installed on the biomass receptacle 105, and the upper press member 110 can engage the seal when the apparatus 100 is in a closed position, thereby forming an effective seal between the upper press member 110 and the biomass receptacle 105.

The apparatus 100 can include a means for compressing the mixture of biomass 400 and solvent 300 while the mixture is positioned in the biomass receptacle 105. The means for compressing the mixture of biomass and solvent can be a pneumatic actuator, a hydraulic actuator, a manual actuator, or any other suitable actuator or combination or actuators.

Since solvents (e.g. ethanol, hexane, acetone) can be flammable, to enhance safety of the solvent recovery apparatus 100, it can be desirable for the apparatus to not contain electrical components that could spark and potentially ignite the solvent. Accordingly, dynamic components of the apparatus, such as the actuator 125, can be pneumatic, hydraulic, or manual to reduce the likelihood of a fire or explosion.

The actuator 125 shown in FIG. 1 is a pneumatic actuator that moves the lower press member 115 vertically when compressed air is delivered to the actuator. In one example, the actuator 125 can be an 8-ton pneumatic jack. In other examples, the actuator can be a 5-10, 8-12, 10-15, 14-20 ton or more than 5-ton pneumatic or hydraulic actuator. The actuator 125 can include a cylinder 128 and a piston 126 that is movable relative to the cylinder.

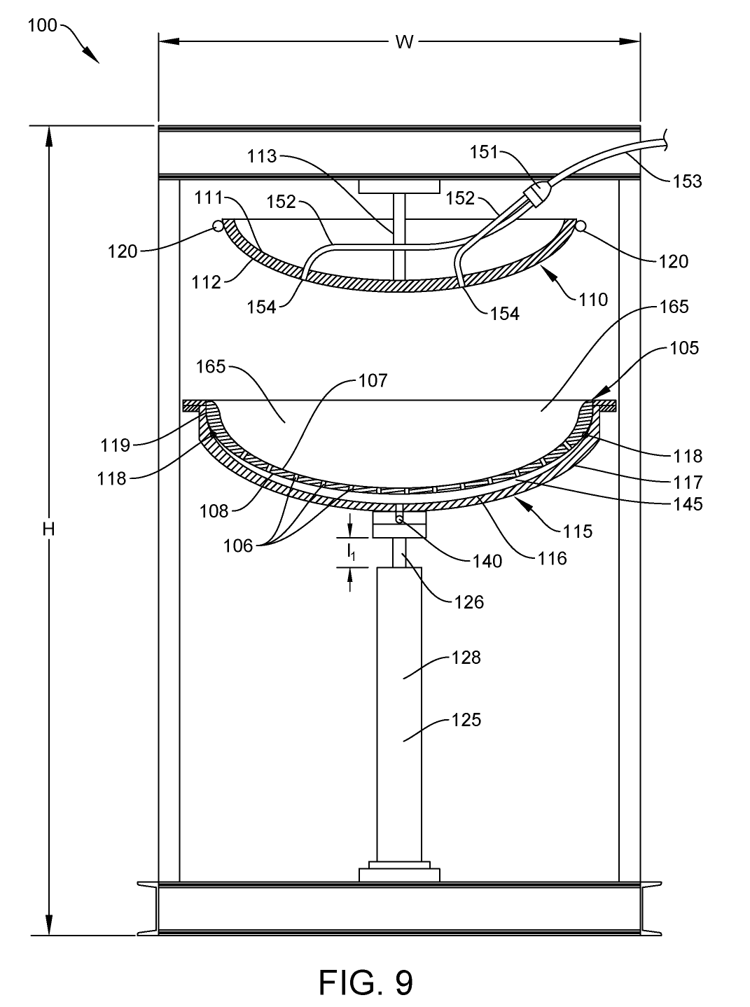

The actuator 125 can be configured to transition the solvent recovery apparatus 100 between an open position (see FIG. 9) and a closed position (see FIG. 10). When in the open position, the length of an exposed portion of the ram 126 can be equal to L₁, as shown in FIG. 9. When the apparatus is in the open position, the biomass receptacle 105 can be positioned a suitable distance below the upper press member 110 to allow a flexible receptacle 160 of biomass 400 to be placed into the biomass receptacle without interference from the upper press member. When in the closed position, the length of an exposed portion of the ram 126 can be equal to L₂, as shown in FIG. 10. When transitioning the apparatus 100 between the open position and the closed position, the exposed portion of the ram can transition from L₁ and L₂.

In addition to being actuated with compressed air, the actuator 125 in FIG. 1 can also be actuated manually by inserting a lever into a lever receiver 127 near the base of the actuator and pumping the lever manually. In one example, an operator can place a container 160 of biomass 400 in the biomass receptacle as shown in FIG. 13 and then use compressed air to transition the solvent recovery apparatus 100 from an open position shown in FIG. 9 to a closed position shown in FIG. 10. Once the apparatus is in the closed position and the biomass receptacle 105 is seated against the seal 120 of the upper press member 110, the operator can then pump the lever manually to increase L₂ and thereby increase the compressive force applied to the biomass material 400. Increasing the compressive force can encourage a mixture of solvent 300, essential oils, waxes, fats, and other lipids (collectively, “solvent mixture”) to flow out of the biomass and then flow downward through the apparatus to the drainage opening 140 where it can be recovered. Increasing the compressive force can also ensure the seal 120 is properly seated against the biomass receptacle 105 prior to operating the pressurized gas injection system 150.

The solvent recovery apparatus 100 can include a lower press member 115. The lower press member (drain basket) 115 can collect solvent that flows through the holes in the biomass receptacle (perforated basket) 105 and funnel the solvent to a drainage opening 140 that is fluidly connected to the vessel (dunk tank) 200, as shown in FIG. 14.

The lower press member 115 can be attached to the actuator 125, as shown in FIGS. 1 and 9. The lower press member (drain basket) 115 can have an inner surface (drainage surface) 116 and an outer surface 117. The lower press member can have a support surface configured to receive and support the biomass receptacle. As shown in FIG. 9, the support surface 119 that receives and supports the biomass receptacle 105 can be an upper portion of the drainage surface 116 of the lower press member 110. As shown in FIG. 9, a seal 118 can provide a gas-tight seal between the lower press member 115 and the biomass receptacle 105. In some examples, the biomass receptacle 105 can be fastened to the lower press member 115, as shown in FIGS. 1-4, to prevent unwanted movement of components during the pressing process.

In FIG. 8, the biomass receptacle 105 is removed from the solvent recovery apparatus 100 to reveal the inner surface 116 of the lower press member 115. The lower press member 115 can include a drainage opening 140 that is located at a low point in the inner surface 116 to utilize gravity when collecting the solvent. The lower press member 115 can have a curved (convex) inner surface 115. In one example, the drainage surface 116 of the upper press member 110 can be hemispherical. Curvature of the drainage surface 116 can encourage the solvent to flow from the holes 106 of the biomass receptacle to the drainage opening 140.

The lower press member 115 can be made of a rigid material capable of withstanding compression without deflecting significantly. In some examples, the lower press member 115 can be made of food-safe material (e.g. stainless steel) and have a thickness of at least 0.25, 0.375, or 0.5 in.

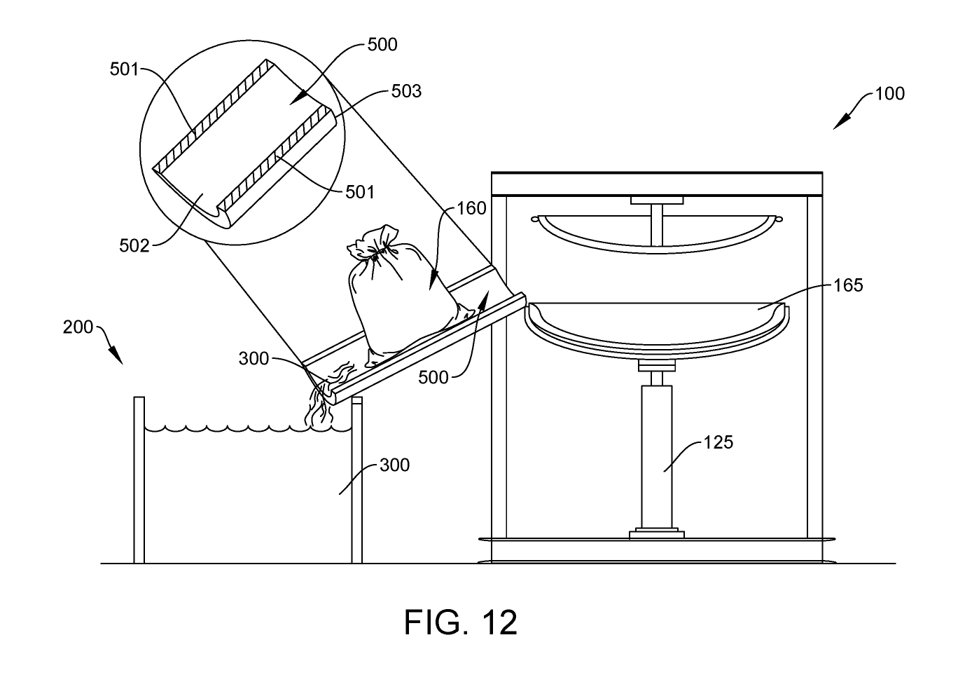

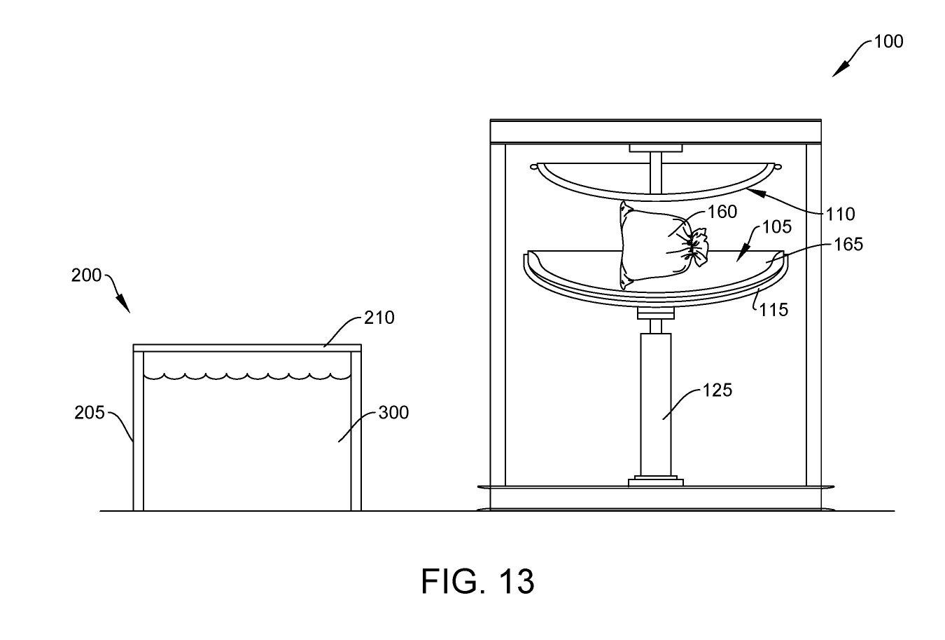

FIGS. 11-14 show an example process for recovering solvent from biomass 400. FIG. 11 shows a step of soaking biomass 400 in solvent to extract essential oils from the biomass. FIG. 12 shows a step of transferring the biomass from the vessel containing solvent (i.e. dunk tank) to a solvent recovery apparatus 100 using a transfer plate 500. FIG. 13 shows a step of placing the biomass, which is wetted or saturated with solvent, in the biomass receptacle of the solvent recovery apparatus 100. FIG. 14 shows the steps of applying compressive force to the biomass by transitioning the apparatus 100 from an open position to a closed position and applying gas pressure via a gas injection system 150.

Prior to the step shown in FIG. 11, the vessel must be filled with solvent 300. During the filling process, solvent can be transferred to the vessel via a solvent supply line 265 fluidly connected to an inlet fitting 270. The inlet fitting 270 and be connected to an inlet port 260 of the vessel 200. A pump can drive flow from a supply tank to the solvent vessel 200. To avoid risk of fire, the pump can operate on compressed air and have no electrical components. In one example, the pump can be a SimpleSpirits air diaphragm distillery pump from VersaMatic. The pump can be compatible with 190-proof ethanol and be ATEX-rated.

FIG. 11 shows a vessel 200 containing solvent 300 located beside an apparatus 100 for recovering solvent. A flexible receptacle 160 containing biomass 400 is shown submerged in solvent within the vessel 200. During the step shown in FIG. 11, the biomass 400 is permitted to soak in the solvent 300 for a predetermined amount of time to allow the solvent to extract desired essential oils from the biomass. The essential oils are able to permeate the material(s) of the flexible receptacle 160 and thereby mix with the solvent in the vessel. The solvent 300 can be any suitable solvent capable of safely extracting desirable materials, such as essential oils, from the biomass. In a preferred example, the solvent can be ethanol (i.e. ethyl alcohol).

The desired amount of time that the biomass 400 is submerged in the solvent 300 depends on, in part, the level of homogenization of the plant matter and the temperature of the solvent . Typically, the colder the solvent, the longer the biomass will need to soak to extract essential oils from the biomass. For example, if the solvent is room temperature, the biomass may only need to soak for about 5 mins, whereas if the solvent is −25 degrees Celsius, the biomass may need to soak for 15 minutes or more. The desired amount of time that the biomass 400 is submerged in the solvent 300 may also depend on desired extraction efficiency and process constraints, such as allowable cycle times.

The flexible receptacle 160 can be a pliable container made of fabric or any other suitable material or combination of materials. In a preferred embodiment, the flexible receptacle 160 can include an interior bag 161 positioned within an exterior bag 162. The interior bag 161 can be made of a breathable, tear-resistant, odor free fabric. The interior bag can be made of food-safe materials, such as woven cotton. The interior bag can be configured to receive the biomass and can be relatively supple and function as a first biomass filter that restricts large biomass particles from exiting the interior bag. In preferred examples, the interior bag is made of a light cotton fabric or light blended cotton and polyester fabric, similar to the material used in wild game bags manufactured by Alaska Game Bags, Inc.

The exterior bag 162 can be made of food-safe materials (e.g. cotton fabric). The exterior bag can be made of a less supple material than the interior bag. In a preferred example, the exterior bag 162 can be made of a durable plain-woven fabric, such as canvas, which can be made of cotton. In other examples, the exterior bag can be canvas made of linen, or hemp. In still other examples, the exterior bag can be made of a durable fabric with a twill weave, such as denim. The exterior bag can function as a second biomass filter that restricts relatively smaller biomass particles (i.e. biomass particles that passed through the interior bag) from passing through the exterior bag and ending up in the vessel 200. In one example, the exterior bag can be made of a material that filters particles larger than about 200 microns.

The material of the exterior bag 162 can be stiffer than the material of the interior bag 161. In one example, the material of the exterior bag can be sufficiently stiff to avoid being drawn into the plurality of holes 106 in the biomass receptacle 105 while pressing the flexible receptacle 160 in the apparatus 100 and applying pressurized gas to the flexible receptacle 160, as shown in FIG. 14. Preventing the material of the exterior bag 162 from being drawn into the plurality of the holes can be desirable, since it can prevent the exterior bag from being damaged (e.g. torn), which could result in biomass escaping the bag and plugging the holes 106 in the biomass receptacle or drainage opening 140 or contaminating the solvent mixture that flows back to the vessel 200 in the step shown in FIG. 14.

The vessel 200 can include a temperature control system to enable chilling of the solvent. In one example, the vessel 200 can be a stainless steel jacketed vessel fluidly connected to a chiller unit that supplies chilled liquid, such as a water-glycol mixture, to isolated passageways in the wall(s) of the vessel. The chilled liquid can be isolated from the solvent within the vessel and, therefore, does not mix with the solvent. While the vessel does not need to be cooled to function, providing cooling is preferred to produce high quality essential oils, which can be more desirable to consumers and more valuable.

In a quick wash ethanol process, the solvent can be capable of removing both essential oils and waxes from the plant matter. Typically, essential oils are desirable and valuable, and waxes are undesirable. It is therefore desirable to operate the extraction process in a way that increases the amount of essential oils recovered and decreases the amount of waxes recovered. Typically, the colder the solvent is, the less wax will be extracted. However, if the solvent is too cold, the extraction efficiency decreases and the time required to extract essential oils will increase, leading to longer processing times, which is undesirable from a commercial processing standpoint. In a preferred example, the solvent 300 in the vessel 200 can be maintained at about −20 to −30 degrees Celsius. This temperature range produces a high yield of desirable essential oils and a low yield of undesirable waxes.

The vessel 200 (dunk tank) can include a lid 210. The lid 210 can be configured to open via a hinge or other suitable mechanism or can be entirely removable. As shown in FIG. 11, the vessel 200 can include an inlet port 260 to allow for filling of the vessel. In one example, the inlet port 260 can be located in a removable lid 210. The inlet port 260 can include an inlet fitting 270 (e.g. a stainless steel tri-clamp fitting) that is configured to fluidly connect the vessel to a solvent supply line 265. The solvent supply line 265 can be made of food-grade materials (e.g. silicone tubing) to avoid contamination of the solvent and to allow the extracted essential oils to comply with food and drug regulations and be used in edible products and personal care products.

After the biomass 400 has been submerged in solvent 300 for a sufficient duration (e.g. 3-5 minutes), the flexible receptacle 160 can be transferred from the vessel 200 (i.e. dunk tank) to the solvent recovery apparatus 100. FIG. 12 shows a step of transferring the flexible receptacle 160 from the vessel 200 to the apparatus 100. A transfer plate 500 can extend from the vessel to the apparatus. In one example, the transfer plate 500 can be a stainless steel plate with a first wall 501 extending upward from a first edge of the plate and a second wall 501 extending upward from a second edge of the plate, as shown in FIG. 12. The transfer plate 500 can include a first lip 502 configured to rest against an inner wall of the vessel 200 and ensure that solvent flowing down the transfer plate is returned to the vessel. The transfer plate 500 can include a second lip 503 configured to rest against the inner surface 107 of the biomass receptacle 105 to keep the transfer plate in place during transfer of the flexible receptacle 160.

A first purpose of the transfer plate 500 is to prevent loss of solvent leaking from the bag 160 by catching the solvent and conveying the solvent back into the vessel. The transfer plate 500 can be arranged at an incline, as shown in FIG. 12, to encourage the solvent to flow back into the vessel due to gravity. The walls of the transfer plate 500 can prevent solvent from flowing off the edges of the plate before it reaches the vessel.

A second purpose of the transfer plate 500 is to reduce a physical strain on an operator who is transferring the bag 160 from the vessel 200 to the press apparatus 100. The transfer plate 50 can support the weight of the bag and its contents during the transfer process, so the operator doesn't need to lift the full weight of the bag and its contents during the transfer process. Instead, since the bag is saturated with solvent, an operator can easily slide the bag along the transfer plate with relatively little physical effort from the vessel 200 to the biomass receptacle 105.

FIG. 13 shows a step of placing the container 160 of biomass 400, which is wetted or saturated with solvent 300, in the biomass receptacle 105 of the solvent recovery apparatus 100. In FIG. 13, the apparatus 100 is in an open position, which provides a sufficient distance between the upper press member 110 and the biomass receptacle 105 to allow an operator to place the flexible receptacle 160 of biomass 400 into the biomass receptacle without interference with the upper press member 110. The flexible receptacle 160 can be positioned in any suitable way within the biomass receptacle. Preferably, the flexible receptacle 160 is placed in the biomass receptacle 105 with its largest surface resting against the inner surface 107 of the biomass receptacle and covering as many of the holes 106 as possible. Covering the holes is desirable, since it causes more of the compressed gas to flow through the bag and the biomass (rather than short-circuiting around the bag) thereby entraining solvent and improving solvent recovery yield.

FIG. 14 shows a step of applying compressive force to the biomass in a flexible receptacle 160 by transitioning the apparatus 100 from an open position to a closed position and then applying gas pressure via the gas injection system 150. Compressing the flexible receptacle 160 of biomass 400 can effectively squeeze solvent from the biomass. The solvent can then flow through the holes 106 in the biomass receptacle and downward to the drainage opening 140. The drainage opening 140 can be connected to a solvent recovery line 230 that transports the solvent back to the vessel 200. As shown in FIGS. 10 and 14, the convex upper press member 110 can nest within the concave biomass receptacle 105, thereby allowing for uniform compression of the wetted biomass 400 in the flexible receptacle 160 while also promoting drainage, by gravitational force, of recovered solvent to the drainage opening 140. Likewise, the biomass receptacle 105 can nest within the lower press member 115 to provide a compact yet durable apparatus that can withstand significant compressive forces and efficiently return solvent to the drainage opening 140.

To increase the amount of solvent recovered from the wetted biomass 400, pressurized gas can be injected proximate a top surface of the flexible receptacle, as shown in FIG. 14. Gas at a pressure of 10-30, 20-40, 30-50, 40-60, 50-70, 60-80, 70-90, or more than 75 psi can be injected into the inner volume 165 of the biomass receptacle. Preferably, air at a pressure of about 15-25 psi is injected proximate a top surface of the flexible receptacle 160 for a first period of time until the flow of solvent through the drainage opening 140 slows or stops, and then air at a pressure of about 60-80 psi is injected proximate a top surface of the flexible receptacle 160 for a second period of time until the flow of solvent through the drainage opening 140 slows or stops. In most instances, the second stage of air injection will completely or nearly completely dry the biomass in the bag 160 and will replace the air in the head space of the 240 of the vessel with clean, dry air, which is desirable to prolong the useful life of the solvent by minimizing exposure to water vapor in non-dry air.

The pressurized gas can flow through the bag 160 and biomass material 400 thereby entraining solvent and transporting the solvent to the drainage opening 140 of the solvent recovery apparatus. More specifically, as shown in FIG. 14, the pressurized gas can flow through an upper surface of the flexible receptacle 160 that is in contact with the lower surface 112 of the upper press member 110, then flow through the wetted biomass 400, then flow through a lower surface of the flexible receptacle that is in contact with the inner surface 107 of the biomass receptacle, then flow through the holes 106 in the biomass receptacle, and then flow to the drainage opening 140. The bag(s) of the flexible receptacle 160 can serve as biomass filters and prevent biomass particles (e.g. particles larger than about 200 microns) from passing through the flexible receptacle and entering the stream of solvent returning to the vessel 200.

As shown in FIGS. 2 and 14, the drainage opening 140 can include a drainage valve 141 to allow an operator to control the flow of solvent back to the vessel 200 via the solvent recovery line 230.

As shown in FIG. 14, the vessel 200 can include a bleed valve 275 to allow for relief of gas pressure from an upper portion of the vessel. The bleed valve 275 can allow the vessel lid 210 to remain closed while applying gas pressure to the biomass 400 and forcing a flow of pressurized gas and solvent from the drainage opening 140 of the apparatus back to the vessel 200. The bleed valve 275 can allow purging of gas from the head space 240 of the vessel, which can improve the flow of solvent from the apparatus 100 to the vessel during operation of the gas injection system 150. This bleed valve 275 allows an operator to keep the lid 210 of the vessel closed while pressing the biomass and passing pressurized gas through the biomass. By keeping the lid 210 on the vessel, it prevents non-dry air from entering the head space 240 of the vessel. By preventing the solvent from absorbing water from the atmosphere, the solvent stays at a higher proof for a longer period of time and, therefore, has a longer useful process life. If non-dry enters the head space 240 of the vessel 200, it can be purged from the head space by flowing compressed air through the gas injection system (which receives clean, dry air from the compressed gas supply system 180) and opening the bleed valve 275 of the vessel 200 to allow gas to escape from the head space 240 of the vessel.

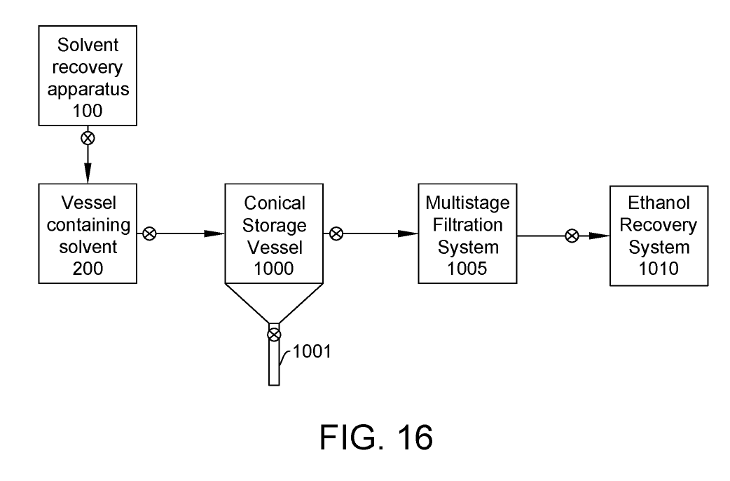

After the solvent is recovered from the biomass 400 in the step shown in FIG. 14, the solvent in the vessel 200 can then be transported to a conical storage vessel 1000, as shown in FIG. 16, where waxes, fats, and other lipids are removed from the solvent.

As shown in FIG. 14, the solvent vessel 200 can include an outlet port 220 near a base of the vessel. The outlet port can include an outlet fitting 221 (e.g. stainless steel tri-clamp fitting) that is fluidly connected to a solvent discharge line 235. The outlet fitting 221 and solvent discharge line 235 can be made of food-grade material(s) to avoid contamination of the solvent. The vessel 200 can include an outlet valve 225 proximate the outlet port 220.

After operating the solvent recovery apparatus 100, the solvent mixture that is recovered from the bag 160 flows back into the vessel 200 via the solvent recovery line 230. The outlet valve of the vessel can then allow the mixture of solvent, essential oils, and waxes in the vessel to be easily transferred via the discharge line 235 to a conical storage vessel 1000 for a winterization process.

To minimize the risk of fire, a non-electric pump, such as a SimpleSpirits air diaphragm distillery pump from VersaMatic, can be used to pump the mixture to the conical storage vessel 1005. The pump can be compatible with 190-proof ethanol and be ATEX-rated. The conical storage vessel can be appropriately sized to receive the volume of solvent being transferred from the solvent vessel 200. The conical storage vessel can be maintained at a temperature of about −10 to −20 Celsius. In one example, the conical storage vessel can be housed in a walk-in freezer.

The purpose of the winterization process is to remove waxes, fats, and other lipids from the mixture of ethanol and essential oils. In some applications, such as when creating distillate, it is desirable to remove waxes, fats, and other lipids to improve the clarity of the distillate.

After the ethanol is pumped into the conical storage vessel 1000, it begins to cool. Waxes, fats, and other lipids precipitate out of solution and fall to the bottom of the conical storage vessel. During precipitation, the wax does not stick to the walls of the conical storage vessel, because its walls are steep. Instead, the waxes migrate into the center of the cone and collect in a spool that extends from the bottom of the conical storage vessel 1000. The spool 1001 can be a long slender member suitable for capturing a volume of waxes, fats, and other lipids that precipitate out of solution. In one example, the spool is a stainless steel tube that attaches to the conical with a tri-clamp fitting and is capped on an opposing end. A valve between the conical storage vessel and the spool can be closed prior to removal of the spool to avoid any loss of solvent. The spool allows for easy removal of precipitate without agitating the solution and causing the waxes to be redissolved.

Once the spool 1001 is removed from the conical storage vessel 1000, the wax can be filtered out using a Buchner funnel. Any amount of ethanol remaining in the spool can be recovered and sent to the ethanol recovery system 1010 where the ethanol is separated from the essential oils.

After the spool has been removed from the conical storage vessel, the mixture in the conical storage vessel can be transferred through a multi-stage filtrations system 1005 and then to an ethanol recovery system 1010, as shown in FIG. 16. In one example, the ethanol recovery system can be a rotary evaporator. The ethanol recovery system can allow the mixture to be separated into solvent and distilled essential oils. The solvent can then be reused in a subsequent extraction process and the distilled essential oils can be used in a wide variety of products.

In one example, a method for recovering solvent 300 from biomass 400 can include providing a flexible receptacle 160 containing biomass and liquid solvent, placing the flexible receptacle containing biomass and liquid solvent into a biomass receptacle 105 where the biomass receptacle has a plurality of openings 106, pressing the flexible receptacle against the biomass receptacle and causing at least a portion of the liquid solvent to exit the flexible receptacle and flow through the plurality of openings 106 in the biomass receptacle 105, and flowing gas through the flexible receptacle containing biomass and liquid solvent to force at least a portion of the liquid solvent to exit the flexible receptacle and flow through the plurality of openings in the biomass receptacle where it can be recovered, as shown in FIG. 14. Flowing gas through the flexible receptacle 160 can include delivering gas at a pressure of 5-100, 10-50, 15-25, or 20 psi proximate a top surface of the flexible receptacle 160. Flowing gas through the flexible receptacle 160 can include delivering gas at a first pressure for a first predetermined duration and delivering gas at a second pressure for a second predetermined duration. The first pressure can be 10-25, 10-30, or 25-40 psi, and the second pressure can be 40-75, 50-100, or 75-125 psi. The first predetermined duration can be 0.5-3, 2-5, or 5-15 minutes, and the second predetermined duration can be 0.5-3, 2-5, or 5-15 minutes. The gas can be selected from a group consisting of nitrogen, argon, air, and carbon dioxide. Pressing the flexible receptacle 160 against the biomass receptacle 105 can include applying a pressure of 10-30, 20-50, 30-50, 40-60, 50-70, 60-80, 70-90, or 80-100 psi to the flexible receptacle. Providing the flexible receptacle can include soaking the flexible receptacle in a vessel of liquid solvent (see FIG. 11) for a predetermined duration and transferring the flexible receptacle from the vessel of liquid solvent to the biomass receptacle using a transfer plate (see FIG. 12). The transfer plate 500 can extend on a decline from the biomass receptacle 105 of a solvent recovery apparatus 100 to the vessel 200 of solvent, allowing liquid solvent that drains from the flexible receptacle during the transfer process to flow on the transfer plate back into the vessel of solvent. Providing the flexible receptacle 160 containing biomass and liquid solvent can include filling an interior bag 161 with biomass 400 and placing the interior bag within an exterior bag, where the exterior bag is made of a durable woven fabric. Pressing the flexible receptacle against the biomass receptacle can occurs prior to, after, or while flowing gas through the flexible receptacle. Preferably, pressing the flexible receptacle 160 against the biomass receptacle can occur prior to and while gas is flowed through the flexible receptacle, as shown in FIG. 14. In this example, the biomass 400 remains under compression while compressed gas flows through the biomass, which can increase the amount of solvent that is recovered from the flexible receptacle.

In another example, a method for recovering solvent 300 from biomass 400 can include providing a flexible receptacle 160 containing biomass and liquid solvent, placing the flexible receptacle of biomass and liquid solvent into a concave biomass receptacle 105 where the concave biomass receptacle has a plurality of openings along a bottom surface of the concave biomass receptacle (see FIG. 13), pressing a convex press member against the flexible receptacle containing biomass and liquid solvent and thereby compressing the biomass and causing a portion of the liquid solvent to exit the flexible receptacle and flow through the plurality of openings in the concave biomass receptacle (see FIG. 14), flowing pressurized gas through the flexible receptacle of biomass and liquid solvent thereby causing a portion of the liquid solvent to exit the flexible receptacle and flow through the plurality of openings in the concave biomass receptacle, and collecting the liquid solvent that flows through the plurality of openings in the concave biomass receptacle. Flowing pressurized gas through the flexible receptacle of biomass 105 and liquid solvent can include flowing gas at a pressure of 5-15, 10-25, 20-40, 30-60, 50-70, 60-80, 70-90, 80-100 psi through the flexible receptacle containing biomass and liquid solvent. Pressing the convex press member 110 against the flexible receptacle 160 of biomass and liquid solvent can include pressing the convex member against the flexible receptacle of biomass and liquid solvent by applying a pressure of 10-30, 20-50, 30-50, 40-60, 50-70, 60-80, 70-90, or 80-100 psi. Providing the flexible receptacle containing biomass and liquid solvent can include providing an interior bag 161 containing the biomass 400 and liquid solvent 300 where the interior cotton bag is positioned within an exterior bag made of canvas, as shown in FIG. 14. Pressing the convex press member 110 against the flexible receptacle 160 of biomass and liquid solvent can occur prior to, while, or after flowing pressurized gas through the flexible receptacle of biomass and liquid solvent.

In another example, a method for recovering liquid solvent 300 from biomass 400 can include providing a biomass receptacle 105 containing a mixture of biomass and liquid solvent, exerting pressure on the mixture of biomass and liquid solvent while the mixture is positioned in the biomass receptacle, flowing pressurized gas through the mixture of biomass and liquid solvent while the mixture is positioned in the biomass receptacle, and collecting the liquid solvent that exits the biomass receptacle.

In one example, an apparatus 100 for recovering liquid solvent 300 from wetted or saturated biomass 400 can include a biomass receptacle 105 having an inner surface 107, an outer surface 108, and a plurality of holes 106 extending through the biomass receptacle from the inner surface to the outer surface where the inner surface of the biomass receptacle defines an inner volume. The apparatus can include a lower press member 115 having a support surface 119 configured to receive and support the biomass receptacle 105, an upper press member 110 having a lower surface 112 configured to seal against a rim surface 109 of the biomass receptacle, and an actuator 125 configured to transition the apparatus from an open position to a closed position, where the inner volume 165 of the biomass receptacle is accessible when the apparatus is in the open position, and where the lower surface 112 of the upper press member 110 seals against the rim surface of the biomass receptacle 105 when the apparatus is in the closed position. The apparatus can include a gas injection system configured to deliver pressurized gas to gas inlets in the lower surface of the upper press member. The gas injection system can be configured to deliver gas at a pressure of 15-80 psi to the gas inlets in the lower surface of the upper press member. The actuator can be configured to apply a compressive force of 10-50, 25-75, 50-100, 75-150, or 100-200 psi between the upper press member and the biomass receptacle when the apparatus is in the closed position. A portion of the upper press member (plunger) 110 can occupy a portion of the inner volume 165 of the biomass receptacle 105 when the apparatus 100 is in the closed position, thereby decreasing available space within the inner volume for a mixture of biomass 400 and solvent and allowing the upper press to compress the biomass and squeeze solvent 300 from the biomass. The lower surface 112 of the upper press member 110 can be a convex surface that occupies a portion of the inner volume of the biomass receptacle when the apparatus is in the closed position, thereby decreasing available space within the inner volume for a mixture of biomass and solvent and allowing the upper press to compress the biomass and squeeze solvent from the biomass. The lower press member can include a drainage surface 116 and a drainage opening 140 fluidly connected to the drainage surface. The apparatus can include a gap 145 between the outer surface 108 of the biomass receptacle 105 and the drainage surface 116 of the lower press member 115 where the gap is configured to permit drainage of liquid solvent from the holes 106 in the biomass receptacle 105 to the drainage opening 140 in the lower press member when the apparatus is in the closed position. At least one of the plurality of holes in the biomass receptacle 105 can have a diameter of about 0.125-0.375 in. The gas injection system 150 can include a gas manifold 151 fluidly connected to one or more gas passageways 152, where the one or more gas passageways are fluidly connected to the gas inlets 154 in the upper press member 110. Each gas passageway 151 can be configured to deliver pressurized gas into the inner volume 165 of the biomass receptacle 105 when the apparatus is in the closed positioned and pressurized gas is supplied to the manifold. The apparatus can include a seal 120 between the lower surface 112 of the upper press member 110 and the rim surface 109 of the biomass receptacle 105. The actuator 125 can be a pneumatic actuator or a hydraulic actuator.

In another example, an apparatus 100 for recovering liquid solvent from biomass can include a press having an upper press member 110, a lower press member 115, and an actuator 125, where the actuator is configured to reduce the distance between the lower press member and the upper press member. The apparatus 100 can include a biomass receptacle 105 positioned between the lower press member 115 and the upper press member 110, where the lower press member and upper press member together are configured to exert a compressive force on the biomass receptacle 105 when the apparatus is in a closed position. The apparatus can include a gas injection system 150 configured to deliver pressurized gas to an inner volume 165 of the biomass receptacle 105 when the apparatus is in the closed position. The apparatus can include a drainage opening 140 configured to allow liquid solvent to flow from the biomass receptacle 105 when the apparatus is in the closed position and a compressive force is exerted on a mixture of biomass and solvent present in the biomass receptacle. The actuator 125 can be configured to reduce the distance between the lower press member 115 and the upper press member 110 by advancing the lower press member toward the upper press member. The actuator 125 can be configured to reduce the distance between the lower press member and the upper press member by advancing the upper press member toward the lower press member. The biomass receptacle 105 can have an inner surface 107, an outer surface 108, and a plurality of openings 106 extending from the inner surface to the outer surface. The inner surface 107 of the biomass receptacle 105 can be hemispherical. The upper press member 110 can have a convex hemispherical surface 112 that is configured to exert a compressive force on a mixture of biomass 400 and solvent 300 when the mixture is located in the concave hemispherical biomass receptacle. The apparatus 100 can include a flexible receptacle 160 having an interior bag and an exterior bag, where the interior bag is configured to insert within the exterior bag, and where the exterior bag comprises a durable fabric.

In another example, an apparatus 100 for recovering liquid solvent 300 from biomass 400 can include a biomass receptacle 105 configured to receive a mixture of biomass and liquid solvent, a means for exerting a compressive force on the mixture of biomass and liquid solvent while the mixture is positioned in the biomass receptacle (see, e.g., FIG. 14), a means for flowing pressurized gas through the mixture while the mixture is positioned in the biomass receptacle (see, e.g., FIG. 14), and a means for collecting the liquid solvent that exits the biomass receptacle (see, e.g., FIG. 14).

The elements and method steps described herein can be used in any combination whether explicitly described or not. All combinations of method steps as described herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The methods and compositions of the present invention can comprise, consist of, or consist essentially of the essential elements and limitations described herein, as well as any additional or optional steps, components, or limitations described herein or otherwise useful in the art.

It is understood that the invention is not confined to the particular construction and arrangement of parts herein illustrated and described, but embraces such modified forms thereof as come within the scope of the claims.

The foregoing description has been presented for purposes of illustration and description. It is not intended to be exhaustive or to limit the claims to the embodiments disclosed. Other modifications and variations may be possible in view of the above teachings. The embodiments were chosen and described to explain the principles of the invention and its practical application to enable others skilled in the art to best utilize the invention in various embodiments and various modifications as are suited to the particular use contemplated. It is intended that the claims be construed to include other alternative embodiments of the invention except insofar as limited by the prior art.

What is claimed is:

1. An apparatus for recovering liquid solvent from biomass, the apparatus comprising:
a biomass receptacle comprising an inner surface, an outer surface, and a plurality of holes extending through the biomass receptacle from the inner surface to the outer surface, the inner surface of the biomass receptacle defining an inner volume;
a lower press member comprising a support surface configured to receive and support the biomass receptacle;
an upper press member comprising a lower surface configured to seal against a rim surface of the biomass receptacle;
an actuator configured to transition the apparatus from an open position to a closed position, wherein the inner volume of the biomass receptacle is accessible when the apparatus is in the open position, and wherein the lower surface of the upper press member seals against the rim surface of the biomass receptacle when the apparatus is in the closed position; and
a gas injection system configured to deliver pressurized gas to gas inlets in the lower surface of the upper press member.

2. The apparatus of claim 1, wherein the gas injection system is configured to deliver gas at a pressure of 15-80 psi to the gas inlets in the lower surface of the upper press member.

3. The apparatus of claim 1, wherein the actuator is configured to apply a compressive force of 10-50, 25-75, 50-100, 75-150, or 100-200 psi between the upper press member and the biomass receptacle when the apparatus is in the closed position.

4. The apparatus of claim 1, wherein a portion of the upper press member occupies a portion of the inner volume of the biomass receptacle when the apparatus is in the closed position, thereby decreasing available space within the inner volume for a mixture of biomass and solvent.

5. The apparatus of claim 1, wherein the lower surface of the upper press member is a convex surface that occupies a portion of the inner volume of the biomass receptacle when the apparatus is in the closed position, thereby decreasing available space within the inner volume for a mixture of biomass and solvent.

6. The apparatus of claim 1, wherein the lower press member comprises a drainage surface and a drainage opening fluidly connected to the drainage surface.

7. The apparatus of claim 6, further comprising a gap between the outer surface of the biomass receptacle and the drainage surface of the lower press member, the gap configured to permit drainage of liquid solvent from the holes in the biomass receptacle to the drainage opening in the lower press member when the apparatus is in the closed position.

8. The apparatus of claim 1, wherein at least one of the plurality of holes in the biomass receptacle has a diameter of 0.125-0.375 in.

9. The apparatus of claim 1, wherein the gas injection system comprises a gas manifold fluidly connected to one or more gas passageways, the one or more gas passageways fluidly connected to the gas inlets in the upper press member.

10. The apparatus of claim 9, wherein each gas passageway is configured to deliver pressurized gas into the inner volume of the biomass receptacle when the apparatus is in the closed positioned and pressurized gas is supplied to the manifold.

11. The apparatus of claim 1, further comprising a seal between the lower surface of the upper press member and the rim surface of the biomass receptacle.

12. The apparatus of claim 1, wherein the actuator is a pneumatic actuator or a hydraulic actuator.

14. An apparatus for recovering liquid solvent from biomass, the apparatus comprising:
a press comprising an upper press member, a lower press member, and an actuator, wherein the actuator is configured to reduce the distance between the lower press member and the upper press member;
a biomass receptacle positioned between the lower press member and the upper press member, wherein the lower press member and upper press member together are configured to exert a compressive force on the biomass receptacle when the apparatus is in a closed position;
a gas injection system configured to deliver pressurized gas to an inner volume of the biomass receptacle when the apparatus is in the closed position; and
a drainage opening configured to allow liquid solvent to flow from the biomass receptacle when the apparatus is in the closed position and a compressive force is exerted on a mixture of biomass and solvent present in the biomass receptacle.

15. The apparatus of claim 14, wherein the actuator is configured to reduce the distance between the lower press member and the upper press member by advancing the lower press member toward the upper press member.

16. The apparatus of claim 14, wherein the actuator is configured to reduce the distance between the lower press member and the upper press member by advancing the upper press member toward the lower press member.

17. The apparatus of claim 14, wherein the biomass receptacle comprises an inner surface and outer surface and a plurality of openings extending from the inner surface to the outer surface.

18. The apparatus of claim 17, wherein the inner surface of the biomass receptacle is hemispherical.

19. The apparatus of claim 14, further comprising a flexible receptacle comprising an interior bag and an exterior bag, wherein the interior bag is configured to insert within the exterior bag, and wherein the exterior bag comprises a durable fabric.

20. An apparatus for recovering liquid solvent from biomass, the apparatus comprising:
a biomass receptacle configured to receive a mixture of biomass and liquid solvent;
a means for exerting a compressive force on the mixture of biomass and liquid solvent while the mixture is positioned in the biomass receptacle;
a means for flowing pressurized gas through the mixture while the mixture is positioned in the biomass receptacle; and
a means for collecting the liquid solvent that exits the biomass receptacle.
